(19) United States
(12) Patent Application Publication
(10) Pub. No.: US 2014/0121445 A1
(43) Pub. Date: May 1, 2014

(54) Intracavitary Brachytherapy Device for Insertion in a Body Cavity and Methods of Use Thereof

(71) Applicant: PF BioMedical Solutions, LLC, Baton Rouge, LA (US)

(72) Inventors: Jonas D. Fontenot, Baton Rouge, LA (US); Michael J. Price, Chapel Hill, NC (US)

(73) Assignee: PF BioMedical Solutions, LLC, Baton Rouge, LA (US)

(21) Appl. No.: 14/065,408

(22) Filed: Oct. 28, 2013

Related U.S. Application Data
(60) Provisional application No. 61/719,431, filed on Oct. 28, 2012.

(51) Int. Cl. A61N 5/10; A61B 1/303; A61B 1/00; A61B 1/05; A61B 1/07

(52) U.S. Cl. CPC A61N 5/1016; A61B 1/05; A61B 1/07; A61B 1/00087; A61B 1/303
USPC 600/6

(57) ABSTRACT

A brachytherapy application device is described, which includes a tandem having a transparent region at its front end, and which is coupled with a fiber-optic illumination means and endoscope. This improved tandem assembly allows the user to guide the tandem into the uterus of a patient in a safer, more reproducible manner with the reduction in occurrence of uterine perforation during tandem advancement and placement.

### CROSS REFERENCE TO RELATED APPLICATIONS

This application claims priority to U.S. Provisional Patent Application Ser. No. 61/719,431, filed Oct. 28, 2012, the contents of which are incorporated herein by reference in its entirety.

### STATEMENT REGARDING FEDERALLY SPONSORED RESEARCH OR DEVELOPMENT

Not applicable.

### REFERENCE TO APPENDIX

Not applicable.

### BACKGROUND OF THE INVENTION

1. Field of the Invention

The inventions disclosed and taught herein relate generally to brachytherapy devices and to methods for cervical brachytherapy treatment using such brachytherapy devices. More particularly, the inventions disclosed herein relate to image-guided brachytherapy devices and image-guided interuterine tandem placement systems having illuminated imaging devices integral with the tandem, for more reproducible and safer placement of the device into the uterus of a subject. Using this device, radioactive therapeutics may be delivered to a region of the uterus, for example, thereby providing for the treatment of cancerous tissue in a safe and reproducible manner.

2. Description of the Related Art

In 2013, approximately 50,000 new cases of cancer of the body of the uterus (uterine corpus) will be diagnosed in the United States (American Cancer Society, Cancer Facts and Figures 2013). Intracavity brachytherapy (ICBT) is an integral part of the treatment regimen for gynecological malignancies, such as cervical, vaginal and endometrial cancer. Approximately 35%, or 17,500 of these cases would be eligible for radiotherapy utilizing ICBT procedures.

Traditionally, many cancers of the uterus are treated with radiation therapy. One manner of delivering such radiation is through an ICBT procedure. In an ICBT procedure, cancerous cells or tissues are irradiated by manually or automatically loading radioactive sources into brachytherapy applicators placed inside the uterine canal during an operative procedure. Between 1996 and 2000, about 84% of these treatments in the U.S. were with low dose rate (LDR) ¹³⁷Cs sources, with the remainder using high dose rate (HDR) ¹⁹²Ir [Eifel, P., et al., Patterns of Radiotherapy Practice for Patients with Carcinoma of the Cervix (1996-1999)]. ICBT may, alternatively or additionally, be administered preoperatively or postoperatively and may be paired with external beam radiotherapy, chemotherapy, or both. Current is applicators used for the treatment of uterine cancer consist of a tandem, which is inserted into the uterus, and a pair of ovoids or colpostats which are inserted into the cervical formixes. Several varieties of these applicators feature fixed intra-ovoid shields designed to reduce complications due to inadvertent irradiation of the rectum, bladder and surrounding tissue. The current practice for proper positioning of the brachytherapy applicator depends on the patient's anatomy and physician's skill.

For a number of years, cervical cancers and carcinomas confined to the uterus, uterine cervix and locally advanced cervical cancers have been optimally managed or treated using radio-chemotherapy. See, for example, Landoni, F., Lancet, Vol. 350, pp. 535-540 (1997); Keys, H. M., et al., N. Engl. J. Med., Vol. 340, pp. 1154-1161 (1999). Major randomized trials have employed external beam radiotherapy followed by a brachytherapy boost, typically with radioactive isotopes such as ¹³⁷Cs, ¹⁹²Ir, and the like. In such brachytherapy, radioactive materials are placed immediately adjacent to the uterine cervix for an amount of time dependent on the activity of the source and the desired level of dose to be delivered to the diseased tissue. Such a procedure requires the placement of an applicator device that consists of three conduits or tubes, where one tube (the tandem) is inserted into the uterus, and the adjoining tubes (the ovoids) are inserted up to the level of the vaginal formixes. The insertion of the tandem applicator tube into the uterine cavity through the cervical os (also referred to as the ostium of the uterus, or the external os) is a challenging procedure to perform. This procedure is often done blindly, by advancing the tandem until the physician senses slight resistance to indicate that the tip of the tandem has approximated the uterine fundus [Barnes, E. A., Int. J. Gynecol. Cancer, Vol. 17, pp. 821-826 (2007)]. The risk of uterine perforation using this technique has been described at rates of 2-14%, and has been postulated to adversely affect patient outcomes [Kim, R. Y., et al., Radiology, Vol. 147, pp. 249-251 (1983); Corn, B. W., et al., Gynecol. Oncol., Vol. 64, pp. 224-229 (1997)].

Brachytherapy may be divided into two main classes: intracavitary and is interstitial. With intracavitary brachytherapy, the radiation sources are placed within a body cavity close to the affected tissue. In interstitial brachytherapy, the radiation sources are implanted within a volume of tissue. Positioning of the radiation sources is an important aspect of brachytherapy. In order to effectively deliver radiation to the target tissue while helping to minimize exposure (and radiation damage) of surrounding healthy or normal tissue, the radiation sources must be properly positioned during the entire course of treatment.

Various types of brachytherapy applicators have been developed for delivering radiation. In the gynecologic field, an exemplary development was the Fletcher-Suit cervical applicator. This applicator consists of a central tube (tandem) and lateral capsules (ovoids or colpostats). The lateral colpostats provide intravaginal positioning while the central tandem traverses the vaginal canal to project into the cervix. Although the Fletcher-Suit applicator has been widely used, maintaining its position in situ can be difficult due to their weight and the difficulty of ensuring a secure connection between the colpostats and tandem. Other brachytherapy applicators have been developed by a variety of entities, e.g., the Miami Vaginal Applicator (Nucletron B V, Veenendaal, N L). However, they can be uncomfortable and/or difficult to insert into the appropriate region of the patient due to their rigidity and incapability of accommodating variations in anatomy, e.g., variations in the size, shape, and orientation of the uterus among patients, or postoperative distortions in anatomy.

Prior art apparatus for such treatment comprises for the radioactively chargeable components a central tubular tandem vaginally insertable longitudinally into the uterine cervix and two ovoids longitudinally locatable at the cervix and laterally positioned between the cervix and the respective vaginal walls. Inasmuch as uterine cervix carcinoma typically spreads to both lateral sides of the cervix, the two longitudinally aligned ovoids are necessarily employed on opposite lateral sides of the cervix-entering, central tandem.

An integral component in determining the dose distribution to be received by the targeted and non-targeted tissues is the positioning of the applicator. ICBT dose distribution planning often involves the use of three dimensional visualization of the targeted areas and surrounding anatomical structures to determine the appropriate position of the implanted applicator in order to maximize a dose delivered to the targeted areas while minimizing dose to healthy tissues. Techniques such as computed-tomography (CT), magnetic resonance (MR), and positron emission tomography (PET) have been employed in the past to generate a three dimensional treatment plan for ICBT procedures. Such techniques have limited the use of shielded ovoids used in ICBT applicators because the shields can interfere with these various methods of planning by distorting images of the implant localization and causing streak artifacts, making a determination of the optimal position of the applicator within the body cavity very difficult to determine.

U.S. Pat. No. 5,562,594 discloses a CT-compatible applicator design (the “Weeks” applicator) that permits CT 3D dosimetry [Weeks, K. J. et al., Int. J. Radiat. Oncol. Biol. Phys.; Vol. 37 (2), pp. 455-463 (1997)]. The Weeks ovoid has tungsten-shielded source carriers which are after-loaded post CT image acquisition. The external shape of the Fletcher-Suit-Declos (FSD) minicolpostat tandem and ovoids system appears to have been the basis for the shape of the Weeks applicator. However, the fixed Fletcher-like shields have been removed and replaced with tungsten shields which are manually loaded in conjunction with the ¹³⁷Cs sources.

The Weeks applicator has been used to develop a technique for improved CT-based applicator localization [Lerma, F. A. and Williamson, J. F., Med. Phys., Vol. 29 (3), pp. 325-333 (2002)]. This study demonstrated that it was possible to support 3D dose planning involving detailed 3D Monte Carlo dose calculations, modeling source positions, shielding and inter-applicator shielding accurately. Nevertheless, the Weeks applicator has several disadvantages. For example, the Weeks applicator is not is adaptable to remote after-loading (loading the radioactive source into the applicator post-insertion and positioning within the body cavity) thereby increasing the radiation exposure from LDR brachytherapy; and, it cannot be used at all for HDR or pulsed dose rate (PDR) applications. In addition, in order to accommodate the after-loading shields, the arms connected to the ovoids are much more bulky than those of a standard FSD applicator. The increased size of the arms makes it more difficult to insert the vaginal packing needed to distance the bladder and rectum from the radiation sources. This added bulk also has a potentially negative impact on the comfort of the patient undergoing treatment.

Another available commercial option is the “Standard CT/MR Applicator” based on a Royal Marsden design from Nucletron Corporation (Sweden). It is designed with special composite tubing which reportedly eliminate distortion on CT or MR images. This applicator is available in different lengths and ovoid diameters to optimize the dose distribution and reduce the mucosal dose. This applicator was not designed for use with any shielding, however, and thus its use results in exposure of the rectum and bladder or other surrounding tissue to high doses of radiation which may lead to clinical complications.

Although all of these devices improve three dimensional localization of the applicator within the patient, none decrease the inherent difficulty of placing the tandem optimally within an orifice of the patient to be treated. Improper tandem placement may increase the risk of tissue perforation or damage during intra-uterine insertion and placement. Compounded by, and in addition to, a lack of reproducible positioning when considering multiple procedures for a single patient, these uncertainties may lead to unpredictable treatment results. Most recently, an ultrasonic approach has been used in some instances to help guide the tandem through the cervix and into the uterus. To do so, a trans-abdominal ultrasound transceiver/receiver is utilized to visualize the tandem shaft once inserted into the uterus. However, given the poor image quality and resolution issues inherent with the is use of ultrasound, such guidance still results in it being difficult for the physician to place the instrument accurately within the patient. Additionally, ultrasonic visualization of the tandem is only possible once the tandem is within the uterus. As such, the use of ultrasonics will not assist with localizing and entering the uterus via the cervical os.

The inventions disclosed and taught herein are directed to efficient and safer applicators having a tandem modified to improve the placement and visualization of the applicator accurately within a patient, as well as methods for the use of such applicators in therapeutic applications.

### BRIEF SUMMARY OF THE INVENTION

The objects described above and other advantages and features of the invention are incorporated in the application as set forth herein, and the associated drawings, related to systems for more safely, efficiently, and reproducibly inserting an applicator assembly into a body cavity of a patient using an image guidance system.

In accordance with an embodiment of the present invention, an applicator apparatus for radioactive therapy of uterine cervix carcinoma is described, the apparatus comprising a tubular tandem having a longitudinally extending finite tandem-length defined by a closed lead-end for insertion through the uterine cervix and a closeable trail-end permitting charging of radioactive material into the tandem leadward portion, the tandem trailward length portion lying substantially parallel to a sagittal-plane; wherein the lead end of the tandem comprises: at least one flexible image sensor disposed within the tandem; one or more discrete optical channels disposed within the tandem; and, a light transmitting means disposed within the interior of the tandem. In further accordance with aspects of this embodiment, at least a portion of the lead-end of the tandem is transparent or semi-transparent.

In accordance with further embodiments of the present invention, methods of treating carcinoma in a patient in need thereof are described, the method comprising the steps of inserting an assembly comprising a tubular tandem and first and second tubular side assemblies through the vaginal orifice of the patient with the tandem extending into the uterus and the first and second side assemblies being positioned against the vaginal wall; and emitting radiation from at least one of the tubular tandem and first and second tubular side assemblies; wherein the step of inserting the tubular tandem includes the steps of integrally associating the lead end of the tandem with an endoscope and illumination means for illuminating the area immediately in front of the lead end of the tandem and activating the endoscope and illumination means prior to or after insertion of the assembly through the vaginal orifice.

In accordance with yet another embodiment of the present invention, a system for delivering radiation therapy to a gynecological tissue is described, the system comprising at least one tandem, optionally modified to house a flexible, fiber optic imaging system such as a camera; a flexible fiber optic scope distal to the tip of the tandem; a light source to provide illumination of the internal anatomy of the subject being treated; an integrated optical/CCD coupler to couple the optical fiber optic scope to an optical imaging system; a computer system to display, record, and archive image data obtained using the system; wherein the lead end of the tandem comprises at least one image sensor disposed within the tandem; one or more discrete optical channels disposed within the tandem; and, a light transmitting means disposed within the interior of the tandem; one or more removable sheaths for housing the one or more brachytherapy applicators during advancement to the target tissue; one or more removable handles for positioning or repositioning the one or more brachytherapy applicators; and instructions for using the one or more brachytherapy applicators. In accordance with aspects of this embodiment, the tip end of the tandem(s) is optically clear and made of an optically transparent or semi-transparent material, and is replaceable or removable by an appropriate attachment system or attachment means.

### BRIEF DESCRIPTION OF THE SEVERAL VIEWS OF THE DRAWINGS

The following figures form part of the present specification and are included to further demonstrate certain aspects of the present invention. The invention may be better understood by reference to one or more of these figures in combination with the detailed description of specific embodiments presented herein.

FIG. 1 illustrates the basic structure of an exemplary brachytherapy apparatus in accordance with embodiments of the disclosure.

FIG. 2 illustrates the position of the apparatus of FIG. 1 within a patient.

FIG. 3 illustrates a fragmented perspective view of an exemplary brachytherapy apparatus in accordance with the present disclosure.

FIG. 3A illustrates a cross-sectional view of the apparatus of FIG. 3, taken along line A-A.

FIG. 4 illustrates a side elevational view of an apparatus in accordance with the present disclosure, superimposed upon the anatomical environment.

While the inventions disclosed herein are susceptible to various modifications and alternative forms, only a few specific embodiments have been shown by way of example in the drawings and are described in detail below. The figures and detailed descriptions of these specific embodiments are not intended to limit the breadth or scope of the inventive concepts or the appended claims in any manner. Rather, the figures and detailed written descriptions are provided to illustrate the inventive concepts to a person of ordinary skill in the art and to enable such person to make and is use the inventive concepts.

### DETAILED DESCRIPTION

The Figures described above and the written description of specific structures and functions below are not presented to limit the scope of what Applicants have invented or the scope of the appended claims. Rather, the Figures and written description are provided to teach any person skilled in the art to make and use the inventions for which patent protection is sought. Those skilled in the art will appreciate that not all features of a commercial embodiment of the inventions are described or shown for the sake of clarity and understanding. Persons of skill in this art will also appreciate that the development of an actual commercial embodiment incorporating aspects of the present inventions will require numerous implementation-specific decisions to achieve the developer's ultimate goal for the commercial embodiment. Such implementation-specific decisions may include, and likely are not limited to, compliance with system-related, business-related, government-related and other constraints, which may vary by specific implementation, location and from time to time. While a developer's efforts might be complex and time-consuming in an absolute sense, such efforts would be, nevertheless, a routine undertaking for those of skill in this art having benefit of this disclosure. It must be understood that the inventions disclosed and taught herein are susceptible to numerous and various modifications and alternative forms. Lastly, the use of a singular term, such as, but not limited to, “a,” is not intended as limiting of the number of items. Also, the use of relational terms, such as, but not limited to, “top,” “bottom,” “left,” “right,” “upper,” “lower,” “down,” “up,” “side,” and the like are used in the written description for clarity in specific reference to the Figures and are not intended to limit the scope of the invention or the appended claims.

Applicants have created an applicator assembly that allows for the visualization is of the insertion of the tandem of the applicator into a body cavity of a patient for therapeutic purposes, wherein the applicator assembly includes a tandem having an illumination means and an imaging element at the terminal end of the tandem.

I. Applicators.

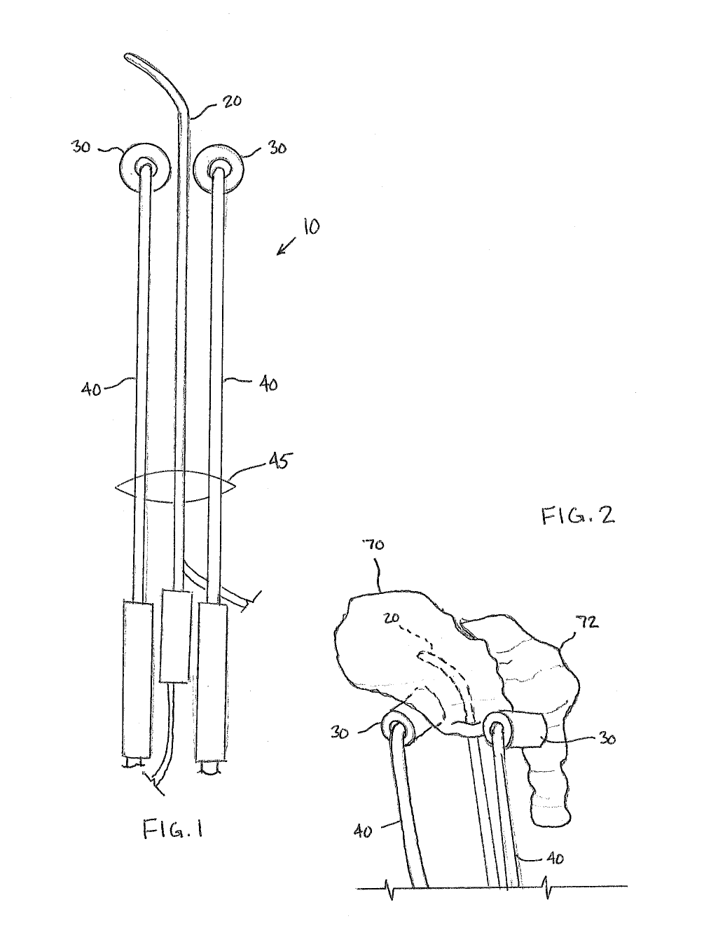

Turning now to the figures, FIG. 1 is depicts the structure of one embodiment of an exemplary applicator of the present invention. This embodiment of the applicator assembly 10 includes a tandem 20 connected to a pivot joint 45 through a tandem arm 25 and, optionally, a pair of colpostats/ovoids 30 which are connected to the pivot joint 45 through a pair of ovoid arms 40. In certain embodiments, the pivot joint 45 serves only to connect the tandem arm 25 to the ovoid arm 40, while in certain other embodiments, the pivot joint 45 functions not only as a connection point but also enables alterations of the angle between the tandem arm 25 and the ovoid arm 40. The tandem 20 and the ovoids 30 are designed to hold one or more therapeutic radioactive source(s), such as an iridium or cesium isotope, during selective irradiation of a patient, such as during a brachytherapy application. In certain embodiments, the tandem arm 25 and/or ovoid arms 40 may be adapted to allow the radioactive source(s) to be loaded through them into the tandem and ovoids, respectively. This can be done after the applicator has been positioned within the body cavity in a process termed “after-loading.”

FIG. 2 depicts a general illustration of the positioning of the applicator assembly 10 of the present invention during treatment of a patient having cervical cancer. The tandem 20 is inserted into the uterus 70 while the ovoids 30 are positioned in the vagina proximal to the external os of the cervix.

During an intracavitary brachytherapy treatment procedure, an applicator 10 of the present invention (as shown in FIG. 1) may be used. Radioactive sources can be placed within the tandem 20 and, optionally, the ovoids 30 as well, in order to provide is a generally spherical- or pear-shaped dose distribution that just surrounds a target volume, with its long axis along the tandem axis. In certain embodiments of the present invention, the radioactive source(s) can be afterloaded into the applicator. In some of these embodiments, the radioactive source is inserted in the ovoids 30 through the ovoid arms 40 and the radioactive source(s) is loaded into the tandem 20 through the tandem arm 25. Prior to the loading of the radioactive source during such procedures, the applicator is often positioned in the body cavity using live image feed from an endoscopic imaging system at the lead-end of the tandem 20, as will be described in further detail below. In accordance with further aspects of the present disclosure, such positioning of the applicator 10 of the present disclosure within a body cavity may also use, as secondary location verification, images from a variety of other, external (e.g., external to the applicator and tandem) secondary sources such as orthogonal X-ray films, CT scans, MR scans, and/or PET scans, can be acquired and used to confirm the location of the applicator 10. These secondary images can also be used to determine and verify that the applicator has been positioned optimally with respect to anatomical location and the dosage of radiation that will be delivered to the targeted area.

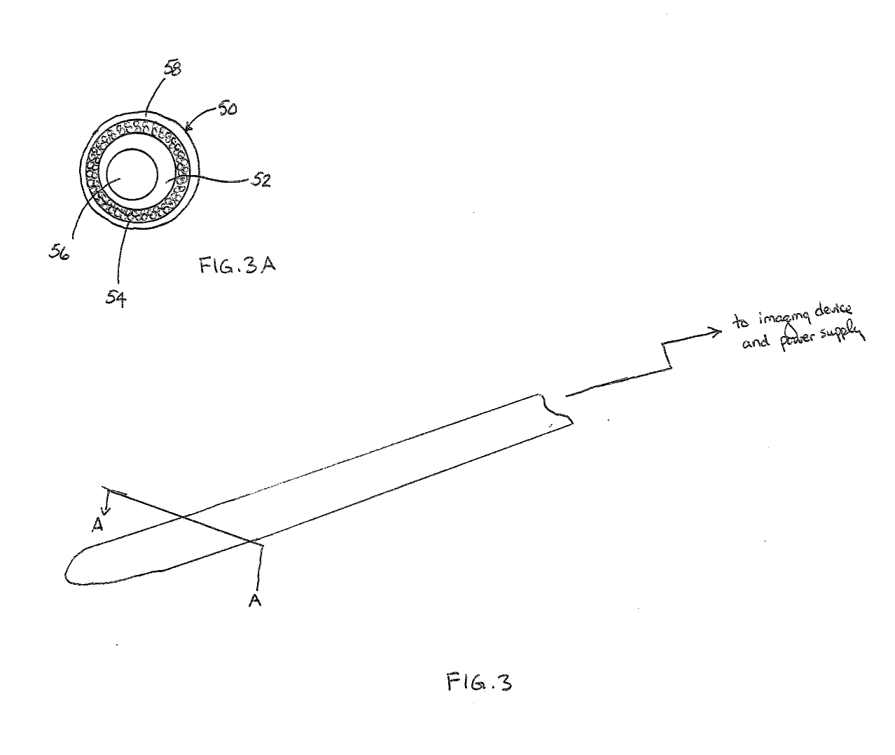

FIG. 3 illustrates a partial, perspective view of tandem 20 of applicator 10 (not shown) in accordance with the present invention. FIG. 3A illustrates a cross-sectional view of the lead-end 21 of tandem 20 in accordance with the present disclosure. These figures will be discussed in combination with each other.

As shown in FIG. 3, tandem 20 is a generally elongated, tubular assembly, having a lead end 21, and containing one (or more) optical endoscopes which are connected to and supported by a handle assembly (not shown), and which is further connected to a power source and an image-viewing device, such as a computer monitor or the like. The majority of the length of the tube of tandem 20 is fabricated from a suitably rigid material, e.g., a metal such as stainless steel or a plastic or is composite material, so as to facilitate the maneuvering of the lead-end (the insertion portion) of the tandem 20 through an orifice into a desired interior cavity of a patient. Preferably, tube 20 is cylindrical as shown, although it may also be formed with some other suitable geometrical cross-sectional shape, including elliptical, oblong/oval and square, as appropriate. Lead end 21 of tandem 20, in accordance with the present disclosure, is preferably fabricated from a translucent material, such as a polymeric, plastic material, so as to allow light (from an illumination means, 54) to pass out through at least the lead end 21, so as to illuminate the placement site viewed with the endoscope 56 contained within the tandem 20. Lead end 21 may be permanently affixed to the tandem arm 25 using an appropriate attachment means, such as an adhesive (chemical bonding) or by a mechanical attachment mechanism, such as screws as by a set of matching screw threads allowing for the mating of the bottom lip of the lead end 21 to the inner surface of the tandem 20.

As shown in the cross-sectional view of the lead-end 21 of tandem 20 in FIG. 3A, the tube body of tandem 20 is generally hollow, having an exterior tubular region 50 and an interior tubular region 52. The annular space between the tubes 50 and 52 may house an illumination source, preferably a flexible illumination source such as an array of optical fibers 54 or a flexible fiber-optic scope, that serve as a means for illuminating the area immediately in front of the front (distal) end of the tube assembly, thus enhancing the viewing by the operator of endoscope 56. Optionally, in accordance with the present disclosure, the illumination source 54 may be a separate tube line running coaxial to endoscope 56, rather than being housed in a channel within the translucent, transparent, or opaque lead-end 21 of the tandem 20. The endoscope 56 typically comprises a lens system comprising one or more high-quality lenses with specific indices of refraction, and preferably being designed to provide a sharply-focused image on an imaging source (e.g., a display monitor on a computer or hand-held device) via an integrated optical/CCD video coupler that is substantially free is of aberrations such as chromatic aberrations. Such an image is provided in real-time or near real-time, and in accordance with select aspects is associated with a video capture software system for viewing the images as well as recording and documenting the tandem placement in vivo. The endoscope is situated at the distal end of the tandem 20, and is preferably locked in place within the interior of tandem 20 by a suitable retaining means, such as (without limitation) a potting compound or by a friction fit. In accordance with some aspects of the present disclosure, the endoscope may include an adjacent, objective lens (not shown) within the interior space 52 of tandem 20, so as to allow for images to be provided to the viewer from offsetting angles, which can be used to convey a sense of depth perception as is often required to provide a three-dimensional observation image. The flexible optic scope is preferably waterproof by way of material or a suitable waterproof coating, and is designed to withstand sterilization of the device.

Preferably, in accordance with select aspects of the invention, a flexible fiber optic scope is used for optical guidance of the apparatus 10. In accordance with one non-limiting example, the scope has an outside diameter of approximately 1.9 mm and a length of approximately 900 mm, and is vinyl coated. The scope has a flexibility allowing for following a bend of up to 45° of the tandem. The direction of view of the scope is 0° along the long axis of the scope, and provides a field of view of up to 45°. This field of view is sufficient for visualization of internal gynecological anatomy but can be expanded by using an appropriately engineered optical tandem tip. The fiber optic bundle within the scope, as detailed herein, provides a resolution of at least 30,000 pixels, providing a large signal-to-noise ratio for intracavity imaging. The light source for the scope, particularly if it is a fiber-optic scope, is one appropriate for the level of object illumination needed. For example, and without limitation, a 10,000 lux, 1.25 W LED will provide adequate illumination, although other alternative light sources such as those providing an illumination of 70,000 lux exhibit substantially ideal illumination for gynecological applications. The assembly can alternatively further include a voltage regulator coupled to the device 10 so as to adjust the degree of is illumination.

The tandem 20 further, preferably includes one or more image relaying means (not shown) at the tail-end 24, which extend the full length of the tandem tube into an assembly or housing that is tied to a power-supply for powering the endoscope and the illumination means within the tandem. This housing further includes an assembly for relaying images from the endoscope to an imaging means in the form of a video unit or video display. The video unit or video display typically comprises a CCD image sensor or the like, having a terminal means for connecting its output signals to a control system (not shown). The terminals of the video units may be connected to an electrical connector assembly, either directly or remotely, that is releasably connected to an electrical cable or the like that leads to an electrical control system (not shown) that is further adapted to process the video image signals received from the endoscope and to apply them to a video monitor or display for image viewing by the operator of the applicator 10 in real-time or near-real-time.

Typically, the optical fiber(s) 54 are connected to a light cable connector assembly at the tail end 28 of the tandem 20, which is in turn connected to a fiber optic cable (not shown) that may be used to couple the optical fiber(s) to a suitable, remote light source.

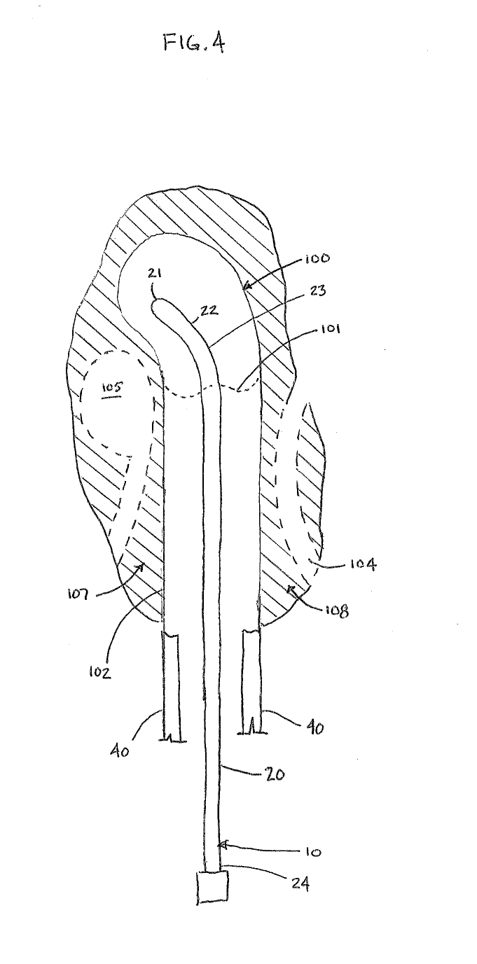

With reference to FIG. 4, the preferred radioactive treatment applicator apparatus 10 of the present invention generally comprises a longitudinally extending tubular tandem 20 having a finite tandem-length parallel sagittal plane (not shown for purpose of clarity) and defined by a closed lead-end 21 and a closeable tail-end 28. When tubular tandem 20 is inserted longitudinally through vaginal orifice 103, lead-end 21 is ultimately positioned inside uterus 100. Tubular tandem 20 at trail-end 28 is closeable e.g. with a removable cap 29, to permit charging of radioactive material (RM) at lead-end 21 as appropriate. Inasmuch as the uterus 100 slopes somewhat transversely forwardly (as seen in FIG. 4), tandem leadward portion 22 also, preferably, slopes forwardly; reference character 23 representing the confluence of leadward portion 22 and the co-sagittal lineal trailward portion 24.

There may further be an adapter member (not shown) slidably surrounding the tubular tandem trailward portion 24 and including releasable arresting means, e.g. a set-screw, for empirically establishing the adapter member at the clinically selected longitudinal position between the tandem lead-end 21 and trail-end 24. In accordance with further aspects of the disclosure, the twin ovoidal assemblies 30, when included with applicator 10, may be removably and pivotably associated with adapter member so as to pivot about transversely extending pivot-axis 45 only whereby the leading-end 41 (containing radioactive material “RM”) is forcibly restrained within a laterally extending mid-plane perpendicular to a sagittal plane and located substantially midway between the transversely separated vasicovaginal (107) and rectovaginal (108) septa.

Each of the two twin ovoidal assemblies 30 comprises an elongated tubular arm 40 having a longitudinally extending finite arm-length less than the tandem-length and defined by a closed leading-end 41 and a closeable trailing-end 48. Each tubular arm is inserted alone longitudinally through vaginal opening 103 until its leading-end 41 is positioned co-elevational with uterine cervix 101, though ultimately movable along the mid-plane from cervix 101 against distensible vaginal wall 102. Respective tubular arms 40 at trailing-end 48 are closeable e.g. with a removable cap 49, to permit the optional charging of radioactive material at the leading-end 41, as appropriate. Each of the tubular arms may also be provided with an ovoid type spacer means removably surrounding arm 40 substantially at its leading-end 41 to maintain some finite spacing between the radioactive material and anatomical parts 100-102.

II. Systems.

In some variations, the systems described herein may optionally include one or more additional apparatus, such as brachytherapy applicators, one or more removable sheaths for housing the applicators and/or apparatus during advancement to the target region of the patient, one or more removable or adjustable handles for the advancement and positioning of the apparatus, or combinations thereof. The sheath, if used, may be of any suitable design and/or material, so long as it is capable of housing and supporting, and in some instances compressing, the apparatus during insertion into the body, and be configured for slidable advancement of the apparatus therethrough, or retraction therefrom. For example, and without limitation, the sheath may be a tubular structure having a rounded tip region.

The systems may also be provided with instructions for using the apparatus. Specifically, the instructions may provide information on how to insert and/or remove the apparatus to or from a patient's body, or provide information regarding loading of an appropriate radiation source into an appropriate region (e.g., a lumen) of the apparatus.

III. Methods of Use.

The fiber-optic based, image-guided tandem insertion apparatus and systems described herein may be used in any area of the body that may benefit from radiation therapy, although as mentioned previously the apparatus are preferably to be used in naturally or surgically created cavities or spaces within the pelvis or abdominal region. With respect to the pelvis, intravaginal, cervical, and intrauterine applications may be useful.

The apparatus described herein can also be used to deliver radiation that is useful in treating any appropriate body tissue in a subject affected by a proliferative condition. Proliferative conditions include tumors, cancers, or other manifestations of abnormal cellular division. For example, and without limitation, the apparatus of the present disclosure may be used, alone or in combination, to treat adenocarcinomas, is carcinomas, leukemias, lymphomas, myelomas, sarcomas, and mixed-type cancers in a subject so affected. Gynecologic cancers such as cervical cancer, endometrial cancer, uterine cancer, ovarian cancer, and vaginal cancer may particularly benefit from visualization and treatment with the apparatus described herein due their conformable and spacing features. Radiation of the vaginal cuff (e.g., after hysterectomy) for endometrial cancer with or without adjuvant pelvic external beam radiation, may also be performed with the apparatus described herein, with appropriate modification. Radiation therapy for proliferative conditions is generally administered over a period of time in partial doses, or fractions, the sum of which comprises a total prescribed dose. For example, about two to about four fractions may be used for vaginal cuff brachytherapy with a total dose of about 10 Gy to about 30 Gy to the target tissue. For cervical cancer, about two to about five fractions may be used with a total dose of about 30 Gy to about 45 Gy to the target tissue. This fractional application takes advantage of cell recovery differences between normal and proliferative tissue, e.g., cancerous tissue, because normal tissue tends to recover between fractions while proliferative tissue tends not to recover or recover at a slower rate.

Treatment planning (dose planning) may occur prior to the initiation of radiation therapy to determine a prescribed dose to be delivered to a volume of the target tissue. In some instances, the prescribed dose may specify a minimum dose to be delivered to a preferred depth outside the treatment cavity (the prescription depth). Other two-dimensional dose prescription regimes may be used as well, e.g., when delivering radiation therapy to the pelvic area. The dose planning process may assess distances from cavity surfaces to skin surfaces or to other radiation sensitive structures (e.g., rectum, bladder, small bowel) and may use these distances in combination with the prescribed prescription depth to determine a dose profile and a dose cloud shape. In this manner, the radiation therapy that is delivered to the target tissue in a subject in need thereof may be configured to provide a pre-determined is dose cloud shape. The dose cloud may be of any suitable shape. For example, the dose cloud shape may be symmetric with respect to the central axis of the applicator or asymmetric with respect to the central axis of the applicator. The bending flexibility of the apparatus described herein, with its highly compressible and conformable surface, combined with its array of spaced peripheral lumens provides for significant patient comfort and dose planning flexibility. Because of the absence of shielding or any metal components in the applicator, three dimensional volumetric-based dose planning with conventional dose planning software (e.g., those available from Varian or Nucleotron) may be readily accomplished with the instant apparatus. This approach includes three-dimensional imaging of the cavity or body region of interest, e.g., by computed tomography (CT), magnetic resonance imaging (MRI), or X-ray, and may be automated. With these three-dimensional dose planning systems, dose planning may be performed more precisely and more accurately, and with a greater characterization of the dose that is being delivered to the target tissue as well as adjacent normal tissue structures. This type of three-dimensional dose planning may also automate the dose delivery, thereby improving dosing accuracy and safety.

The brachytherapy applicators may be inserted and advanced in any suitable manner. In some variations, the brachytherapy applicators are collapsed outside the body from an initial expanded configuration to an unexpanded configuration. The applicator in its unexpanded configuration is then inserted, e.g., within a body cavity, and advanced to the target tissue. After appropriate positioning, the applicator may then be expanded into its expanded deployed configuration. A sheath may be employed when inserting the brachytherapy applicators, but need not be. When a sheath is used, the brachytherapy applicators may be preloaded in the sheath. Robotic insertion of the apparatus described herein is also contemplated.

Given that the apparatus described herein are generally compliant and lack the typical rigid components, they are generally less traumatic to position and secure in the patient. Taking this into consideration, it is contemplated that reduced anesthesia is and/or sedation will be needed for placement of these apparatus. In some variations, removable or permanent internal stiffener elements may be employed to facilitate applicator placement. The stiffeners may reside in one or more lumens or may be located elsewhere within the elongate body. In other variations, a hygroscopic laminaria or other gradual cervical dilating device to dilate the cervix prior to inserting a tandem and/or other components of the applicator may be used to facilitate proper applicator placement with reduced anesthesia and/or sedation requirements.

When the apparatus is associated with brachytherapy (such as when a subject is in need of radiation therapy), the radiation sources for targeted delivery may then be placed within the apparatus by any suitable method. For example, the radiation sources may be afterloaded, either by hand (manual afterloading) or by a machine (automatic remote afterloading) after the brachytherapy applicators are positioned. In other variations, hot loading may be employed. With hot loading, the brachytherapy applicator contains the radiation sources at the time of placement into the subject in need of radiation therapy. The radiation therapy that is subsequently delivered by the radiation sources may provide radiation therapy in a pre-determined dose cloud shape, as previously stated.

A proof of concept experiment to test the effectiveness and ergonomics of the tandem apparatus of the current disclosure was conducted in a healthy, human volunteer, in accordance with standard protocols. The volunteer was neither pregnant nor had undergone a hysterectomy. The sterilized apparatus was connected to an imagine device, and the device inserted into the vagina. Once the apparatus 10 was inside the vagina, the imaging and illumination features were activated, and the apparatus was advanced toward the os with no other guidance means other than the optical guidance within the tandem. During the assessment of tandem placement, the patient did not report experiencing discomfort, and no perforation of the uterine cavity or other anatomical area by the tandem was observed.

In addition to image-guided tandem placement, the position and/or orientation of intracavitary ovoids can be validated using the assembly of the present disclosure. To this end, a fiber optic scope may be inserted into a rigid, thin-walled cylinder with an internal diameter just larger than the diameter of the scope (e.g., 2 mm). The clinician can then insert the scope into the vagina of the patient so as to view the ovoid placement in vivo.

Other and further embodiments utilizing one or more aspects of the inventions described above can be devised without departing from the spirit of Applicant's invention. For example, the devices described herein may be used in combination with other therapy devices, including those that utilize ultrasound, CT, or MRI to assist in guiding tandem placement for brachytherapy applications in a subject. Further, the various methods and embodiments of the methods of manufacture and assembly of the system, as well as location specifications, can be included in combination with each other to produce variations of the disclosed methods and embodiments. Discussion of singular elements can include plural elements and vice-versa.

The order of steps can occur in a variety of sequences unless otherwise specifically limited. The various steps described herein can be combined with other steps, interlineated with the stated steps, and/or split into multiple steps. Similarly, elements have been described functionally and can be embodied as separate components or can be combined into components having multiple functions.

The inventions have been described in the context of preferred and other embodiments and not every embodiment of the invention has been described. Obvious modifications and alterations to the described embodiments are available to those of ordinary skill in the art. The disclosed and undisclosed embodiments are not intended to limit or restrict the scope or applicability of the invention conceived of by the Applicants, but rather, in conformity with the patent laws, Applicants intend to fully protect all such modifications and improvements that come within the scope or range of equivalent of the following claims.

What is claimed is:

1. An applicator apparatus for radioactive therapy of uterine cervix carcinoma, the apparatus comprising:
a tubular tandem having a longitudinally extending finite tandem-length defined by a closed lead-end for insertion through the uterine cervix and a closeable trail-end permitting charging of radioactive material into the tandem leadward portion, the tandem trailward length portion lying substantially parallel to a sagittal-plane;
wherein the lead end of the tandem comprises:
at least one image sensor disposed within the tandem;
one or more discrete optical channels disposed within the tandem; and,
a light transmitting means disposed within the interior of the tandem.

2. The apparatus of claim 1, wherein a least a portion of the lead-end of the tandem is transparent or semi-transparent.

3. The apparatus of claim 1, further comprising two ovoidal assemblies each comprising an elongate tubular arm having a directionally longitudinal finite arm-length less than the tandem-length and defined by a closed leading-end and a closeable trailing-end permitting charging of radioactive material into the arm lead-length portion,
each ovoidal assembly at the arm trail-length portion being removably and/or pivotably attachable to the adapter member whereby the leading-end of the respective arms is free to move laterally away from the tandem leadward length portion.

4. A method of treating carcinoma in a patient in need thereof, the method comprising the steps of:
inserting an assembly comprising a tubular tandem and first and second tubular side assemblies through the vaginal orifice of the patient with the tandem extending into the uterus and the first and second side assemblies being positioned against the vaginal wall; and
emitting radiation from at least one of the tubular tandem and first and second tubular side assemblies;
wherein the step of inserting the tubular tandem includes the steps of integrally associating the lead end of the tandem with an endoscope and illumination means for illuminating the area immediately in front of the lead end of the tandem and activating the endoscope and illumination means prior to or after insertion of the assembly through the vaginal orifice.

5. The method of claim 4, wherein the method of treating carcinoma in a patient includes delivering a radiation therapy to a gynecological tissue in a subject in need thereof using the apparatus of claim 1.

6. The method of claim 5, wherein the radiation therapy is delivered by a radiation source.

7. The method of claim 6, wherein the radiation source comprises a radioactive liquid, an x-ray source, a radiation seed, or combinations thereof.

8. The method of claim 6, wherein the radiation source comprises radionuclides selected from the group consisting of cesium, iridium, iodine, cobalt, palladium, strontium, yttrium, gold, ruthenium, californium, and combinations thereof.

9. The method of claim 6, wherein the radiation source is loaded into the central catheter, at least one peripheral catheter, or a combination thereof, using an afterloader.

10. The method of claim 6, wherein the radiation source is used to treat a gynecological cancer.

11. The method of claim 10, wherein the gynecological cancer is selected from the group consisting of cervical cancer, endometrial cancer, uterine cancer, ovarian cancer, and vaginal cancer.

12. The method of claim 5, wherein the target tissue is the uterus, the cervix, or the vaginal cuff.

13. A system for delivering radiation therapy to a gynecological tissue, the system comprising:
one or more gynecological brachytherapy applicators, the brachytherapy applicators comprising a tubular tandem having a longitudinally extending finite tandem-length defined by a closed lead-end for insertion through the uterine cervix and a closeable trail-end permitting charging of radioactive material into the tandem leadward portion, the tandem trailward length portion lying substantially parallel to a sagittal-plane;
wherein the lead end of the tandem comprises:
at least one image sensor disposed within the tandem;
one or more discrete optical channels disposed within the tandem; and,
a light transmitting means disposed within the interior of the tandem;
one or more removable sheaths for housing the one or more brachytherapy applicators during advancement to the target tissue;
one or more removable handles for positioning or repositioning the one or more brachytherapy applicators; and
instructions for using the one or more brachytherapy applicators.

14. The system of claim 13, comprising a plurality of brachytherapy applicators.

15. The system of claim 13, wherein the plurality of brachytherapy applicators vary in length, shape at the distal end, number of peripheral passages and catheters, initial expanded diameter, unexpanded diameter, expanded deployed diameter, or a combination thereof.

16. The system of claim 39, further comprising one or more radiation sources.

17. The system of claim 16, wherein the one or more radiation sources comprise a radioactive liquid, an x-ray source, a radiation seed, or combinations thereof.
